(19) United States
(12) Patent Application Publication
(10) Pub. No.: US 2020/0265949 A1
(43) Pub. Date: Aug. 20, 2020

(54) ANXIETY DETECTION IN DIFFERENT USER STATES

(71) Applicant: Holland Bloorview Kids Rehabilitation Hospital, Toronto (CA)

(72) Inventors: Azadeh KUSHKI, Toronto (CA); Akshay Sainag Reddy PULI, Toronto (CA)

(21) Appl. No.: 16/276,208

(22) Filed: Feb. 14, 2019

(51) Int. Cl. G16H 40/63

(52) U.S. Cl. CPC G16H 40/63

(57) ABSTRACT

Methods and systems are described for providing output based on detection of anxiety in a subject. Output is provided, dependent on an anxiety indication that represents a current or expected level of anxiety in the subject. A physiological signal is received, representing physiological information from the subject. A context signal is also received. A user state detector determines a current user state from a plurality of possible user states, based on the context signal. An interactive multiple model (IMM) filter is used to determine, using the physiological signal, a statistical prediction of anxiety in each of the possible user states. An anxiety detector is used to output the anxiety indication, based on a weighting of the statistical predictions using the determined current user state.

### FIELD

The present disclosure relates to methods and systems for detection of anxiety in the context of different user states, including states (e.g., presence of motion) that cause physiological arousal not related to anxiety.

### BACKGROUND

Anxiety is a significant clinical concern in autism spectrum disorder (ASD) due to its negative impact on physical and psychological health. For example, up to 85% of children with ASD experience clinically-significant symptoms of anxiety [3]. Anxiety in ASD is a significant clinical concern as it can further exacerbate core symptoms and increase functional impairments [4]. Treatment of anxiety in ASD is a challenge. Traditional approaches to anxiety treatment rely on self-awareness of anxiety symptoms—an area of significant difficulty in ASD [5]. This is a barrier to treatment as symptom awareness is key to timely and effective application of management strategies.

Physiological signals offer an opportunity to address the above challenge. In particular, physiological signals collected through non-invasive and commercially-available wearable sensors can provide a real-time, objective, and language-free measure of anxiety states [6]. A technical challenge in developing an anxiety detection system is modelling baseline physiological characteristics of users and identifying significant changes from this baseline that correspond to anxiety states. To this end, supervised and unsupervised learners such as K-Nearest Neighbours (KNN), Regression Tress (RT), Bayesian Network (BNT), support vector machines (SVM), and adaptive filters have been used to detect anxiety states using physiological signals [7], [15]-[18]. A limitation of these approaches is that physiological arousal is not specific to anxiety and may be associated with other user states such as physical activity. This results in false positives which hinder the real-world operation of existing anxiety detection systems.

Thus, there exists a need to provide an approach for real-time detection of anxiety in different user states, including user states that may cause physiological arousal not related to anxiety.

### SUMMARY

The present disclosure describes examples for real-time detection of anxiety, which may also mitigate against false positives due to physical activity effects. The examples disclosed herein may enable realization of physiological anxiety detection methods and systems in naturalistic settings and/or in a user's day-to-day life. Examples of the present disclosure may be implemented using wearables and mobile computing platforms, including currently available consumer electronics.

In some examples, the present disclosure describes an approach that uses a multiple model Kalman-like filter to account for different user states. For example, in order to account for user motion, the multiple model Kalman-like filter proposed may integrate heart rate and accelerometry signals, by tracking user heart rate under different motion assumptions, and determining the appropriate model for anxiety detection based on user motion conditions. Evaluation of an example implementation found a reduction in false positives compared to the state-of-the-art, and an overall arousal detection accuracy of 91%.

In some aspects, the present disclosure describes a system for providing output based on detection of anxiety in a subject. The system includes an output device for providing output dependent on an anxiety indication, the anxiety indication representing a current or expected level of anxiety in the subject. The system also includes a memory and a processor coupled to the output device and the memory. The processor is configured to execute computer-executable instructions to cause the system to: receive at least one physiological signal, from a first sensor, the physiological signal representing physiological information from the subject; receive at least one context signal; implement a user state detector to determine a current user state from a plurality of possible user states, based on the at least one context signal; implement an interactive multiple model (IMM) filter to determine, using the physiological signal, a respective statistical prediction of anxiety in each of the plurality of possible user states; and implement an anxiety detector to output the anxiety indication, based on a weighting of the respective statistical predictions using the determined current user state.

In any of the above, the instructions, when executed, may further cause the system to: implement a feature extractor to: extract the at least one physiological feature from the at least one physiological signal, the at least one physiological feature being affected by the level of anxiety in the subject; and extract the at least one context feature from the at least one context signal, the at least one context feature being relevant to determination of the current user state. The user state detector may determine the current user state based on the at least one context feature extracted from the at least one context signal. The IMM filter may determine the respective statistical predictions based on the at least one physiological feature extracted from the at least one physiological signal.

In any of the above, the instructions, when executed, may further cause the system to implement the feature extractor to: extract the at least one physiological feature by calculating a trend using a first defined smoothing window length; and extract the at least one context feature by calculating a moving standard deviation using a second defined smoothing window length.

In any of the above, the at least one physiological signal may include a heart rate signal, the at least one context signal may include an acceleration signal, and the plurality of possible user states may include a first user state where the user is in motion and a second user state where the user is not in motion.

In any of the above, the system may also include a heart rate monitor for generating the heart rate signal, and an accelerometer for generating the acceleration signal.

In any of the above, the instructions, when executed, may further cause the system to implement the user state detector to: determine the current user state using a modified Kalman filter.

In any of the above, the instructions, when executed, may further cause the system to implement the IMM filter to: determine the respective statistical prediction of anxiety using a respective modified Kalman filter matched to each respective possible user state.

In any of the above, at least one of the at least one context signal may be received from a context sensor of the system.

In any of the above, at least one of the at least one context signal may be received from an external system.

In any of the above, the output device may be a display screen and the provided output may be a visual output that is responsive to the current or expected level of anxiety in the subject.

In any of the above, the system may be implemented in a portable electronic device.

In any of the above, the system may be implemented in a wearable electronic device.

In any of the above, the system may be implemented in a virtual reality device.

In any of the above, the instructions may be executable by the processor via cloud computing.

In any of the above, the instructions may be executable by the processor via an application programming interface (API) on a server.

In some aspects, the present disclosure describes a method, implemented in an electronic device, for providing output based on detection of anxiety in a subject. The method includes: receiving at least one physiological signal, from a first sensor coupled to the electronic device, the physiological signal representing physiological information from the subject; receiving at least one context signal; implementing, in the electronic device, a user state detector to determine a current user state from a plurality of possible user states, based on the at least one context signal; implementing, in the electronic device, an interactive multiple model (IMM) filter to determine, using the physiological signal, a respective statistical prediction of anxiety in each of the plurality of possible user states; implementing, in the electronic device, an anxiety detector to output an anxiety indication, based on a weighting of the respective statistical predictions using the determined current user state, the anxiety indication representing a current or expected level of anxiety in the subject; and providing output, via an output device of the electronic device, dependent on the anxiety indication.

In any of the above, the method may also include implementing, in the electronic device, a feature extractor to: extract the at least one physiological feature from the at least one physiological signal, the at least one physiological feature being affected by the level of anxiety in the subject; and extract the at least one context feature from the at least one context signal, the at least one context feature being relevant to determination of the current user state. The user state detector may determine the current user state based on the at least one context feature extracted from the at least one context signal. The IMM filter may determine the respective statistical predictions based on the at least one physiological feature extracted from the at least one physiological signal.

In any of the above, the at least one physiological signal may include a heart rate signal received from a heart rate sensor coupled to the electronic device, the at least one context signal may include an acceleration signal received from an accelerometer coupled to the electronic device, and the plurality of possible user states may include a first user state where the user is in motion and a second user state where the user is not in motion.

In any of the above, the user state detector may determine the current user state using a modified Kalman filter.

In any of the above, the IMM filter may determine the respective statistical prediction of anxiety using a respective modified Kalman filter matched to each respective possible user state.

### BRIEF DESCRIPTION OF THE DRAWINGS

Reference will now be made, by way of example, to the accompanying drawings which show example embodiments of the present application, and in which:

FIG. 1 is a chart illustrating the effect of physical activity on heart rate;

FIG. 2 shows example equations for a single-model modified Kalman filter for anxiety detection;

FIG. 3A is a block diagram illustrating an example disclosed system for anxiety detection in different user states;

FIG. 3B is a block diagram illustrating another example disclosed system for anxiety detection, where the user states include user motion;

FIGS. 4A-4B show example equations for a multimodal Kalman filter for anxiety detection in different user states;

FIG. 5 is a block diagram of an example processing unit implementing an example system for anxiety detection;

FIG. 6 illustrates the experimental protocol for an example study of anxiety detection;

FIG. 7 is a chart representing the average heart rate across all participants in an example study of anxiety detection;

FIG. 8 is a chart representing the effect of the acceleration smoothing window length parameter on performance of an example anxiety detection system;

FIG. 9 is a chart representing the effect of the innovation window width parameter on performance of an example anxiety detection system;

FIG. 10 is a chart representing the effect of the detection threshold parameter on performance of an example motion detector in an example anxiety detection system;

FIG. 11 is a chart representing the effect of the RR smoothing window length parameter on performance of an example anxiety detector in an example anxiety detection system;

FIG. 12 is a chart representing the effect of the innovation window length parameter on performance of an example anxiety detection system;

FIG. 13 is a chart representing the effect of the offset parameter on performance of an example anxiety detection system;

FIG. 14 is a chart representing the effect of the transition probability parameter on performance of an example anxiety detection system;

FIG. 15 is a chart representing the effect of the detection threshold parameter on performance of an example anxiety detector in an example anxiety detection system; and

FIG. 16 illustrates an example operation of an example disclosed anxiety detection system.

Similar reference numerals may have been used in different figures to denote similar components.

### DESCRIPTION OF EXAMPLE EMBODIMENTS

The present disclosure describes examples for detection of anxiety in users with autism spectrum disorder (ASD), however it should be understood that the present disclosure is not limited to use in this population. For example, the present disclosure may be useful for detection of anxiety in any application where it may be useful or therapeutic to provide information, including feedback to the user, about the user's anxiety level. The present disclosure describes examples in which detection of anxiety is performed in different user states, such as a user state where there is user motion. It should be understood that the different user states that may be accommodated by the disclosed methods and systems are not limited to user motion detected using motion sensors (e.g., accelerometers), and may include other user states that may be detected using information from other sensors.

To help in understanding the present disclosure, a brief discussion of the challenges in detection of anxiety is first provided. The autonomic nervous system (ANS) controls involuntary visceral functions of the body, including cardiac activity. The ANS is divided into parasympathetic and sympathetic pathways, which are associated with arousal and dampening of the autonomic responses, respectively. These subsystems exert excitatory and inhibitory control over the heart muscle, and their combined effect can be observed through measurement of heart rate.

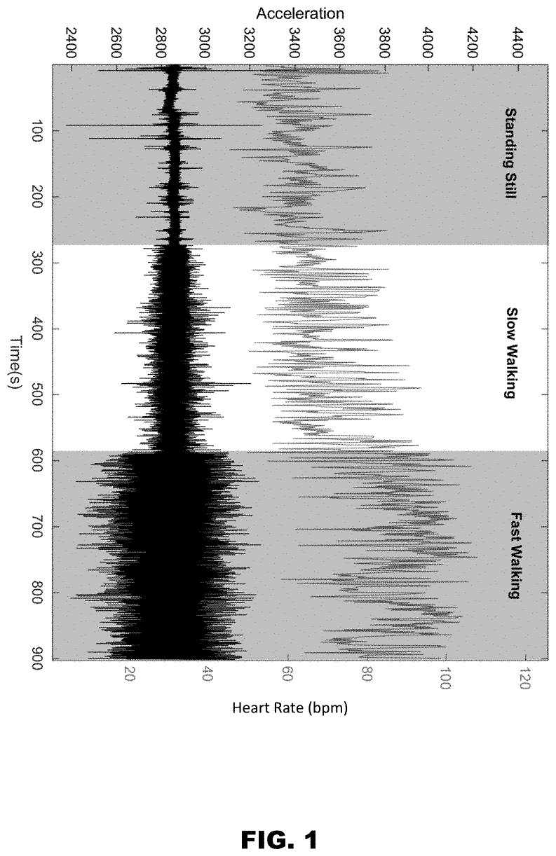

States of anxiety are generally associated with sympathetic dominance and thus increased heart rate [19], [20]. This type of response, however, is not unique to anxiety. For example, physical activity is also associated with sympathetic dominance. This is the body's physiological response to meet the energy needs of the body resulting from muscle activity. To illustrate this, FIG. 1 shows the effect of physical activity (as indicated by acceleration—in this case represented as unit-less values that correlate directly with magnitude of acceleration) on heart rate. As can be seen from FIG. 1, heart rate is increased with increased motion.

This non-specificity of ANS arousal can lead to false positives and decreased performance of anxiety detection systems in presence of user motion. This is a limitation in naturalistic environments where users are often mobile. Although this challenge has not been directly addressed, a handful of studies have incorporated accelerometry signals in feature vectors used in supervised classification for a stationary user [7], [8], [21]. Other studies have used accelerometry signals to classify user activity. For example, a Kalman-like state estimator was proposed in [22] to detect four activity classes (sit, stand, walk, run). However, none of these studies have addressed how to use accelerometry signals together with physiological signals to improve the specificity of arousal detection.

As well, the above-noted approaches use supervised learning algorithms that require training data collected under different physical and cognitive states. This is typically cumbersome for users and impractical when a large number of movement situations are present.

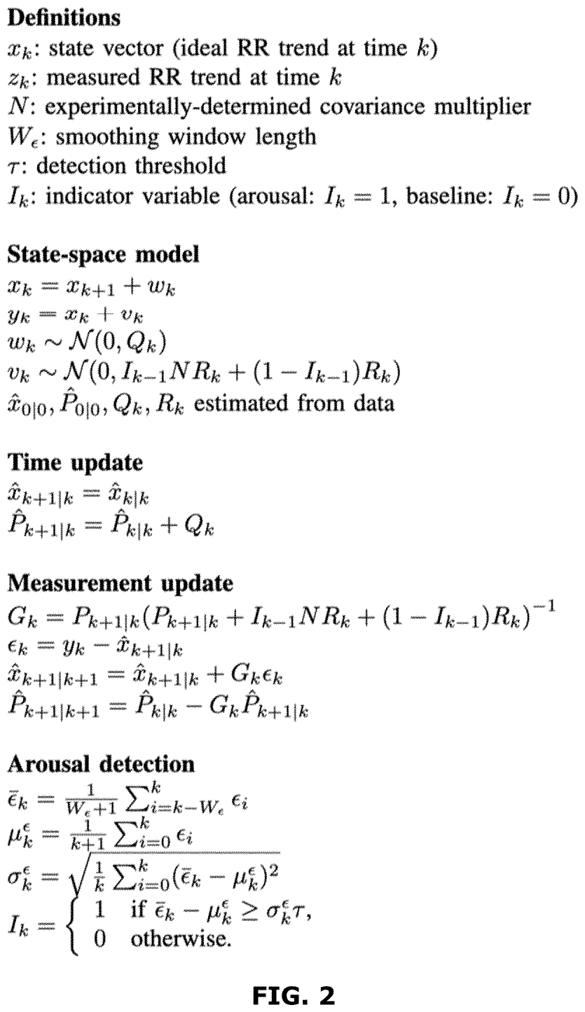

An approach for unsupervised and real-time detection of anxiety is described in [12] and in U.S. Pat. No. 9,844,332, which uses a Kalman filter-based approach for anxiety detection. FIG. 2 shows some example equations for implementing this approach. The unsupervised approach eliminates the need for cumbersome initial training as well as retraining to adapt to changing user and environmental conditions. However, this approach does not explicitly account for the effects of physical activity, or other user states that cause physiological arousal not specific to anxiety.

In the present disclosure, the Kalman filtering approach is extended into an interactive multiple model (IMM) filter (e.g., implemented using modified Kalman filters). The disclosed methods and systems provide unsupervised anxiety detection using multiple model filtering. In examples discussed below, heart rate is assumed to be the hidden state of a dynamical system that operates in one of two or more possible modes, each mode reflecting a different user state (e.g., a rest mode that assumes a lack of motion; and a motion mode that assumes the presence of physical activity, and hence higher baseline heart rate). Each of these modes is associated with a respective modified Kalman filter. The IMM filter includes all the modified Kalman filters for the different modes and also combines the state estimates from each modified Kalman filter together using mixing probabilities.

Data received from sensors (including at least a physiological sensor) provides information for determining physiological arousal and for determining the user state. Baselines for each model are established, and deviations from these baseline models are used to change states appropriately and detect anxiety.

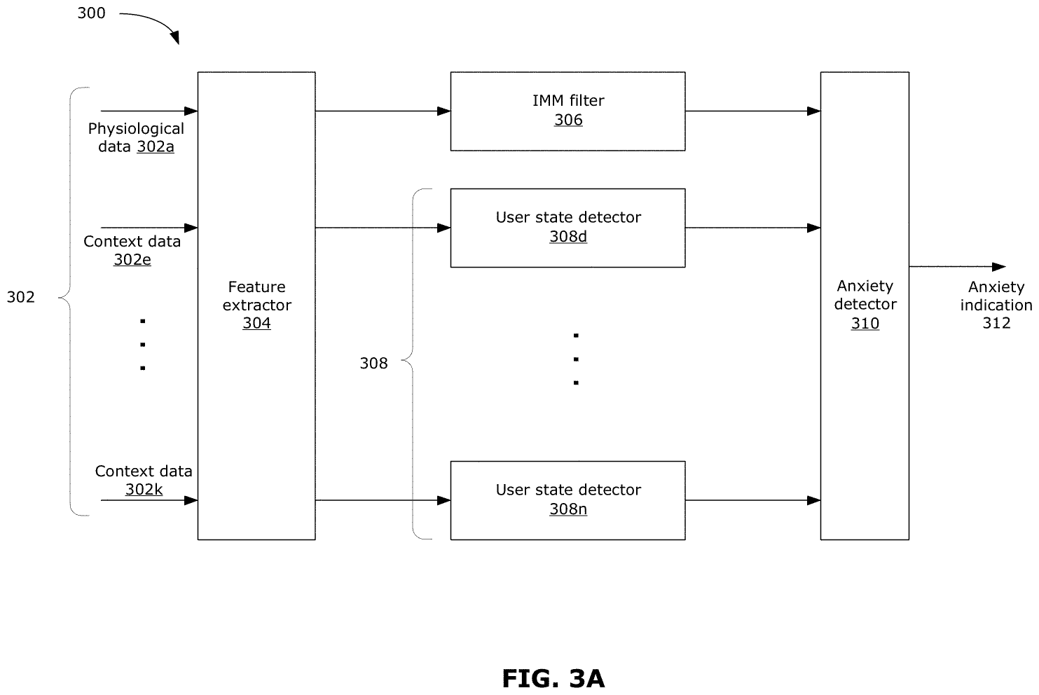

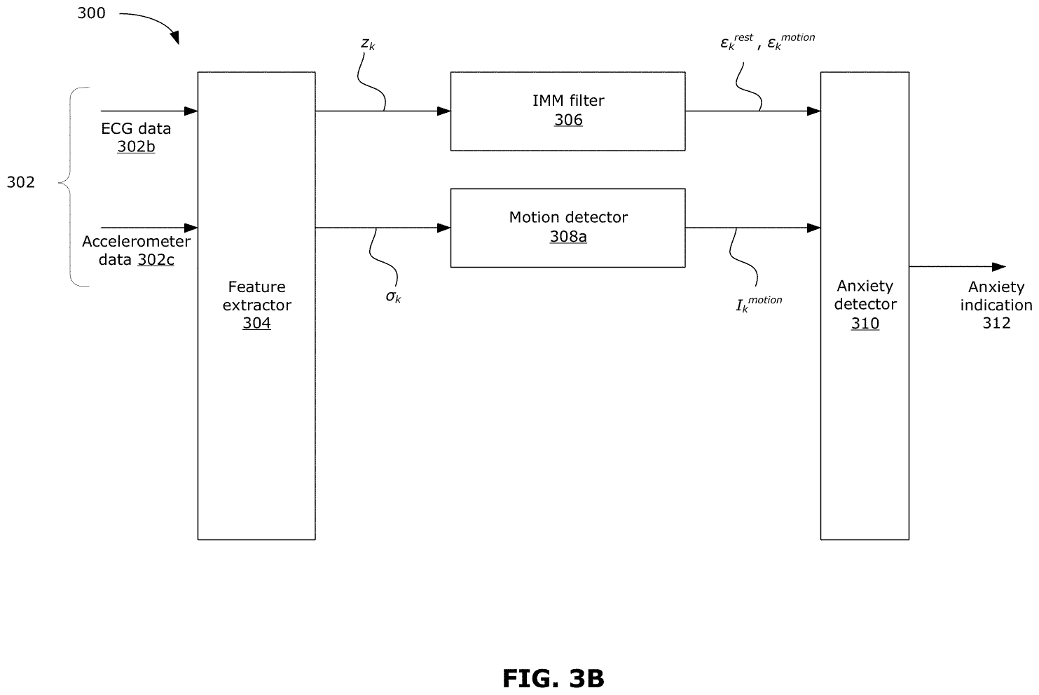

A simplified block diagram illustrating an example disclosed anxiety detection system 300 is shown in FIG. 3A. The anxiety detection system 300 may be implemented using software, hardware, or a combination thereof. As will be discussed further below, the anxiety detection system 300 may be implemented by or as part of another computing system or processing unit. The anxiety detection system 300 is configured to account for a plurality of defined user states. The occurrence of a particular user state is determined using user state detector(s). An IMM filter calculates a statistical prediction for anxiety, for all defined user states. An anxiety detector receives the calculated probabilities and the determinations of user states, and processes this information together to output an anxiety indication that accounts for the user being in one of the defined user states.

The system 300 receives as input data 302 received from one or more sensors. The input data 302 includes at least one set of physiological data 302a (e.g., data received from a physiological sensor), which provides physiological information for determining arousal. The input data 302 also includes one or more sets of context data 302e-302k (e.g., data received from other sensors, which may or may not be physiological sensors), which provides information for determining a user state. In some example, context data 302e-302k may be obtained from a non-sensor source, such as an external database or a software application. This type of context data 302e-302k may include, for example, data received from a calendar application, a clock and/or a GPS application, among other possibilities. Such context data 302e-302k may provide information about user state, such as whether the user is scheduled to be at a gym, whether the user is sleeping vs. awake, or whether the user is in a warm climate vs. cold climate.

It should be noted that, in some examples, contextual information for determining a user state may also be determined using data from a physiological sensor (e.g., a body temperature sensor may be used to determine a hot or cold user state), thus there may be overlap between the type of data that is considered physiological data 302a and the type of data that is considered context data 302e-302k. For simplicity, input data 302 may be used to generally refer to both physiological data 302a and context data 302e-302k.

The input data 302 is processed by a feature extractor 304. The feature extractor 304 is used to process the raw input data 302 to extract features that can be used in state-space models by the user state detector(s) 308 and the IMM filter 306. The feature extractor 304 may, for example, process the raw input data 302 to remove noise or transitory signals. The feature extractor 304 may also quantify the raw input data 302 and/or label the raw input data 302 in a way that can be used in state-space models.

In some examples, the feature extractor 304 may perform different processing on each input data 302, and may extract different features from each input data 302. For example, the feature extractor 304 may perform low-pass filtering on input data from a temperature sensor to remove noise and transitory signals, based on the expectation that temperature changes are relatively gradual.

On the other hand, input data from an accelerometer may be processed using a smoothing window (e.g., as discussed in the example of FIG. 3B below) because accelerometer data is expected to be more fast-changing. The feature extractor 304 may extract different features based on the different characteristics of different input data. For example, heart rate data contains unique physiological characteristics, such as occurrence of the QRS complex, which can be used by the feature extractor 304 to quantify cardiac activity (e.g., as discussed in the example of FIG. 3B below). On the other hand, context data may be categorized by the feature extractor 304 based on the user context indicated by the context data. For example, the feature extractor 304 may classify time data as being “day” or “night”. It should be understood that different ways of processing input data and extracting features may be used, within the scope of the present disclosure.

Although the present disclosure refers to feature(s) extracted from the input data, in some examples it may not be necessary to extract feature(s) from the input data 302, and the user state detector(s) 308 and/or IMM filter 306 may process at least some of the input data 302 directly.

The output of the feature extractor 304 is received by one or more user state detectors 308d-308n (generically referred to as user state detector 308). Each user state detector 308 is configured to detect the occurrence of a particular user state, based on feature(s) of the input data 302. In some examples, each user state detector 308 may be implemented using a modified Kalman filter, and determines a binary indicator for a particular user state based on one extracted feature.

In the present disclosure, the modified Kalman filter may be based on the algorithm shown in FIG. 2. The modified Kalman filter allows for incorporation of different states (e.g., baseline and motion, in the case of motion detection), unlike a traditional Kalman filter that assumes a single state (e.g., baseline only). In the modified Kalman filter, the baseline state model is updated using the feature(s) of the input data 302 when the deviation from the baseline is not significant (e.g., falling within a predicted noise model). When the feature(s) of the input data 302 deviates significantly from the baseline (e.g., falling outside the predicted noise model), this is considered to be indicative of the non-baseline state (e.g., motion state, in the case of motion detection) and the feature(s) of the input data 302 is not used to update the baseline state model. Instead, the output is an indicator of the non-baseline state.

Generally, the modified Kalman filter updates the baseline state model using a first weighting of the feature(s) when the feature(s) has a value within a predicted noise model, and updates the baseline state model using a lesser second weighting (which could be zero) of the feature(s) when the feature(s) has a value outside of the predicted noise model.

An example detailed implementation of the user state detector 308, using a modified Kalman filter, is discussed below with respect to FIG. 3B showing an example embodiment for motion detection. In some examples, a user state detector 308 may be implemented using other approaches aside from a modified Kalman filter. For example, depending on the feature being analyzed by the user detector 308, the user detector 308 may determine occurrence of a particular user state by comparing the feature against a predefined threshold (e.g., a sleep state is determined if the time is later than a threshold time), or determining whether the feature fits into a particular category (e.g., a motion state is determined if the location is categorized as an exercise location), among other possibilities. Each user state detector 308 may use different approaches to determining the occurrence of a respective user state.

The IMM filter 306 is configured to implement a plurality of modified Kalman filters (each matched to a respective defined user state), to calculate a statistical prediction of anxiety in each possible user state. In this example, the IMM filter 306 further includes a model to mix the state estimates outputted from the plurality of modified Kalman filters, as discussed further below. The IMM filter 306 accepts as input feature(s) extracted from the physiological data 302a, and calculates a statistical prediction of anxiety for each possible user state. An example calculation of statistical prediction is the calculation of an innovation. Generally, in statistical analysis, the innovation is calculated as the difference between an observed value of a variable at a given time, and an optimal forecast value of that variable. The calculated innovation thus may be used as an indication of whether there is a deviation from baseline, to determine the presence of anxiety.

The IMM filter 306 addresses the problem of false positives, discussed above with respect to existing approaches for anxiety detection. In a prior approach that uses a modified Kalman filter (e.g., represented by the equations of FIG. 2), it is assumed that the system follows a single linear-Gaussian model. This assumption limits the performance of the system in cases where changes in user state (e.g., motion) may cause significant deviations from the baseline model. This results in growing filter error, and thus possible false anxiety detections. To mitigate against this challenge, the IMM filter 306 in the disclosed example system 300 uses a jump-linear model to track the physiological features under different user states, and information from context data (e.g., acceleration data) is used to select the appropriate model for anxiety detection at any time point. An example detailed implementation of the IMM filter 306 is discussed below with respect to FIG. 3B showing an example embodiment.

The anxiety detector 310 receives, from the IMM filter 306, the calculated innovation for each possible user state, and applies weights to the innovations using the determined user states outputted from the user state detector(s). The weighted innovation is then used to calculate mean and covariances for anxiety detection. An example detailed implementation of the anxiety detector 310 is discussed below with respect to FIG. 3B showing an example embodiment.

A detailed example implementation of the system 300 will be discussed with reference to FIG. 3B, which shows an example embodiment of the system 300 for detection of anxiety in the presence of possible user motion. The ability to accurately detect anxiety when the user is in a state of motion is of particular interest. Previous attempts at detection of anxiety have used supervised approaches, in which, using machine learning approaches (e.g., such as support vector machine, K-nearest neighbour, and decision tree algorithms) models have been trained to detect arousal of the ANS based on cardiac activity and other physiological signals [8], [12], [30]-[32]. However, most of these algorithms have been evaluated based on data collected while the subject is at rest. The presence of user motion challenges the operation of these systems as physical activity is also associated with ANS arousal. This can result in false positives and performance degradation. In the present disclosure, an example of the anxiety detection system 300 is described below, in which the disclosed unsupervised approach to anxiety detection is used to account for ANS changes related to physical activity.

It is well-known that cardiac activity increases during states of ANS arousal associated with both physical activity and anxiety. However, it has not been clearly established clear if physical activity could give rise to non-anxiety-specific arousal that could be falsely detected as anxiety. It also has not been clearly established if there can be detectable anxiety-related increase in arousal during physical activity. In an example study, discussed further below, it has been found that heart rate does in fact increase significantly in response to anxiety tasks, even in presence of physical activity. This further motivates the need for anxiety detection methods that can accurately detect anxiety even in presence of user motion.

In the example of FIG. 3B, it is assumed that the system 300 can operate in one of two user states: motion or no motion. An accelerometry signal (e.g., from a tri-axial accelerometer) is used to modulate how the IMM switches between states and to determine the threshold for anxiety detection in each mode. In this example, the system 300 enables reduction of false anxiety detections, by accounting for physical activity-related arousal. Further, the system 300 enables detection of anxiety-related arousal during physical activity.

In the example of FIG. 3B, the input data 302 includes electrocardiogram (ECG) data 302b (as an example of physiological data 302a), and tri-axial accelerometer data 302c (as an example of context data 302b). The input data 302 are processed through the feature extractor 304 to obtain the heart rate and accelerometry feature time series, which are then processed by a motion detector 308a (as an example of user state detector 308) and the IMM filter 306. The anxiety detector 310 receives output from the IMM filter 306 and the motion detector 308a to produce the anxiety indication 312 (e.g., a binary value).

In the example of FIG. 3B, the feature extractor 304 processes the accelerometer data 302c and the ECG data 302b as follows. The smoothed moving standard deviation of the accelerometer data 302c is used to compute the acceleration vector σₖ as follows:

$${σ}_{k} = \sqrt{\frac{1}{2 {ω}_{A} + 1} {\sum }_{i = k - {ω}_{A}}^{k + {ω}_{A}} {( {υ}_{i} - {μ}_{{υ}_{i}} )}^{2}} \qquad ( 1 )$$

where k is the time index and μᵥᵢ is the mean of vᵢ over the window of interest. The signal vᵢ is the magnitude of the acceleration data in the x, y, z directions at time i (e.g., bandpass filtered between 0.25 and 5 Hz, and re-sampled to 5 Hz). The window length w_{A} may be selected based on experimental or empirical testing.

The ECG data 302b is quantified based on the length of RR intervals, for example extracted using the Pan-Tompkins algorithm [23] and re-sampled uniformly at 5 Hz. The RR time-series is used to compute a slowly varying trend zₖ at time k defined as [12]:

$${z}_{k} = \frac{1}{{ω}_{RR} + 1} {\sum }_{i = k - {ω}_{RR}}^{k} {RR}_{i} \qquad ( 2 )$$

where the window size w_{RR} is the smoothing window length, which may be determined using experimental or empirical testing. The results of the processing by the feature extractor 304 are then provided for further processing by the IMM filter 306 and the motion detector 308a as described below.

The motion detector 308a processes the accelerometry feature time series, σₖ, to produce a binary indicator Iₖ^{motion} (where 0 indicates no motion, 1 indicates motion). The motion detector 308a may be implemented using any suitable algorithm. For example, a modified Kalman filter may be used with the following state-space model:

xₖ=xₖ₋₁+wₖ (3)

σₖ=xₖ+vₖ (4)

where xₖ is a state variable modelling the evolution of user motion, σₖ is acceleration vector defined in Equation 1, and wₖ and vₖ are zero-mean Gaussian system and measurement noises, respectively, determined as in [12]. Other approaches may be used for implementing the motion detector 308a, including supervised methods (e.g., using machine learning algorithms) or other unsupervised methods.

As noted above, the IMM filter 306 in this example uses a jump-linear model to track the RR-series under rest and motion conditions, and makes use of accelerometer data to select the appropriate model for anxiety detection at any time point.

A jump-linear model is defined with two modes M ∈{rest,motion}. In particular, it is assumed that mode switching (mode jump process) is a Markov process with transition probabilities defined a priori as:

p^{ij}≡P(Mₖ=j|Mₖ₋₁=i) (5)

For simplicity, the transition matrix is modeled as:

$$P ≡ ( \begin{matrix}p & 1 - p \\ 1 - p & p\end{matrix} ) \qquad ( 6 )$$

To track the state, the IMM filter 306 uses a filter bank comprised of two modified Kalman filters, each matched to rest or motion modes, respectively. Each of these filters assumes a linear-Gaussian state-space model defined below:

χₖ^{M}=χₖ₋₁^{M}+wₖ^{M} (7)

zₖ=χₖ^{M}+vₖ^{M} (8)

where the state estimate x^{M}ₖ is the “ideal” slow varying RR trend at time k for model M ∈{rest,motion}, and zₖ^{M} is the observed RR trend defined in Equation 2. The process noise wₖ^{M} and measurement noise vₖ^{M} are assumed to be independent, zero-mean Gaussian noise with variances Q^{M}ₖ and Rₖ^{M}, respectively. Each mode-matched filter tracks the baseline RR series under the assumption of no anxiety, allowing the anxiety detection under both rest and motion states. The initial condition for the rest-matched filter is computed from the data, while the initial condition for the motion filter is assumed to be the rest state plus an offset (the offset may be selected experimentally, as discussed further below).

The estimates from each filter are computed following the approach of the IMM filter [24]. This approach is based on combining state estimates and covariances from each filter using estimated model probabilities. These mixing probabilities may be computed using the following equation:

$${μ}_{k - 1 \vert k - 1}^{M \vert U} = \frac{{p}^{MU} {μ}_{k - 1}^{M}}{{\sum }_{M} {p}^{MU} {μ}_{k - 1}^{M}} \qquad ( 9 )$$

These probabilities are used to compute the mixed initial conditions for each filter using the filter's estimate from the previous iteration, according to the equation:

$$\begin{matrix}{\hat{x}}_{k - k \vert k - 1}^{0 M} = {\sum }_{U} {\hat{x}}_{k - 1 \vert k - 1}^{U} {μ}_{k - 1 \vert k - 1}^{U \vert M} & ( 10 ) \\ {P}_{k - 1 \vert k - 1}^{0 M} = {\sum }_{U} {μ}^{U \vert M} ( {P}_{k - 1 \vert k - 1}^{U} + {( {\hat{x}}_{k - 1 \vert k - 1}^{U} - {\hat{x}}_{k - 1 \vert k - 1}^{0 M} )}^{2} ) & ( 11 )\end{matrix}$$

where ∪∈{rest,motion}. Based on the estimates and their covariances, the prediction that contributes to the innovation ϵₖ^{M} and its covariance Sₖ^{M} is computed for each model, as follows:

ϵₖ^{M}=yₖ−\hat{χ}_{k|k-1}^{M} (12)

Sₖ^{M}=\hat{P}_{k|k-1}^{M}+Iₖ₋₁^{arousal}Rₖ+(1−Iₖ₋₁^{arousal})N Rₖ. (13)

Finally, to prepare for the next iteration of the filter, the probability of each mode being correct, μ^{M}ₖ, is estimated using each filter's likelihood function, as follows:

$${μ}_{k \vert k}^{M} = \frac{𝒩 ( {ϵ}_{k}^{M} , {S}_{k}^{M} ) ( {\sum }_{U} {p}^{UM} {μ}_{k - 1}^{M} )}{{\sum }_{U} 𝒩 ( {ϵ}_{k}^{M} , {S}_{k}^{M} ) ( {\sum }_{U} {p}^{UM} {μ}_{k - 1}^{M} )} \qquad ( 14 )$$

The innovation signal ϵₖ^{M} quantifies the amount of deviation between the observation and the mode-matched baseline. The output, Iₖ^{motion}, of the motion detector 308a is used to choose the innovation that will be used to determine the presence of arousal. In particular,

ϵₖ=(1−Iₖ^{motion})ϵₖ^{rest}+Iₖ^{motion}ϵₖ^{motion} (15)

The innovation εₖ is then used by the anxiety detector 310 to compute mean and covariances for anxiety detection as in [12]:

$$\begin{matrix}{μ}_{k}^{ϵ} = \frac{1}{N + 1} {\sum }_{i = 0}^{k} {ϵ}_{i} , & ( 16 ) \\ {\overline{ϵ}}_{k} = \frac{1}{{W}_{n} + 1} {\sum }_{i = k - {W}_{n}}^{k} {ϵ}_{i} & ( 17 ) \\ {σ}_{{ϵ}_{k}} = \sqrt{\frac{1}{k} {\sum }_{i = 0}^{k} {( {\overline{ϵ}}_{i} - {μ}_{k}^{ϵ} )}^{2}} , & ( 18 )\end{matrix}$$

where Wₙ is a moving average window. The arousal indicator Iₖ^{arousal} is determined using the following equation:

$${I}_{k}^{arousal} = \{ \begin{matrix}1 & if {\overline{ϵ}}_{k} - {μ}_{k}^{ϵ} \geq {σ}_{k}^{ϵ} {τ}_{k} , \\ 0 & otherwise .\end{matrix} \qquad ( 19 )$$

The anxiety detector 310 outputs the arousal indicator Iₖ^{arousal} as the anxiety indication 312.

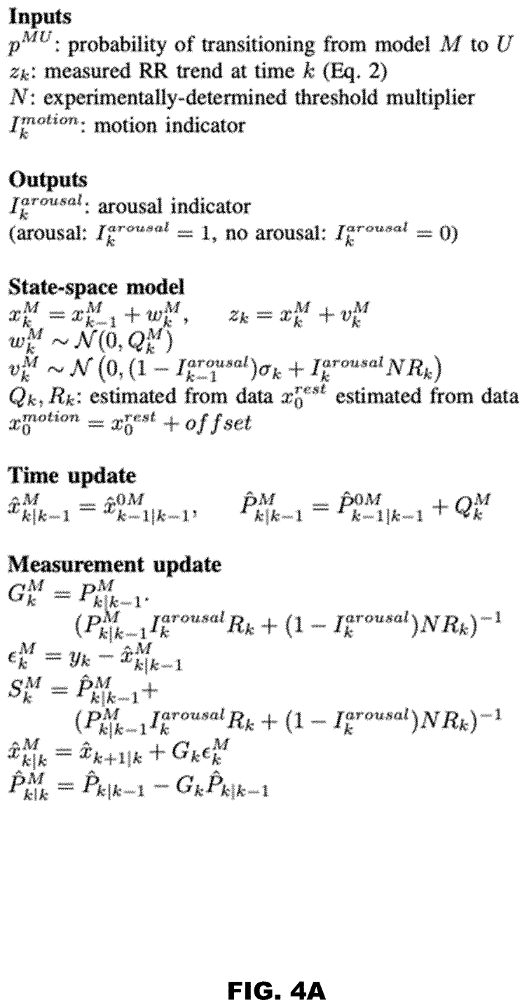

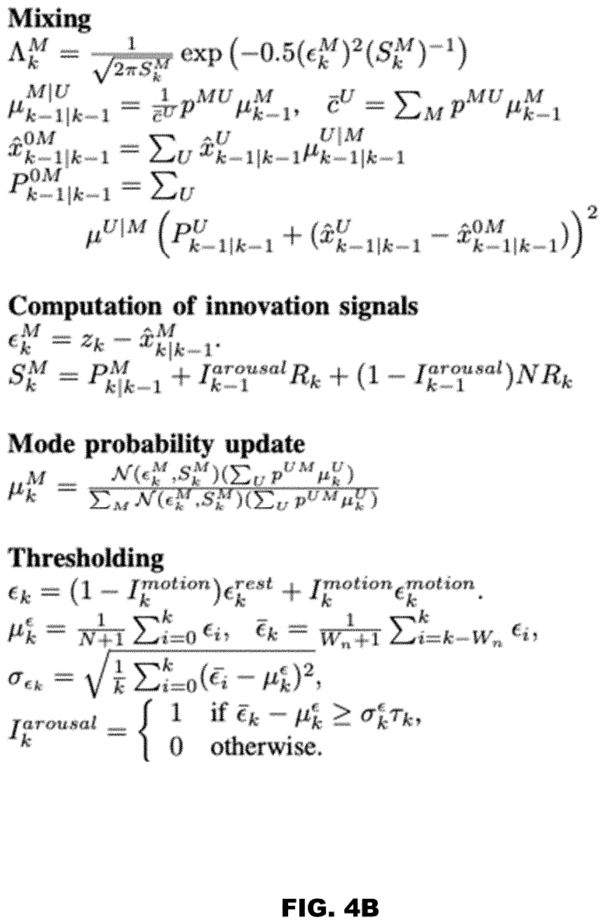

FIGS. 4A-4B show equations summarizing the disclosed example system 300, in the case where user motion is taken into account. In particular, the equations of FIGS. 4A-4B may be used to implement the IMM filter 306 with a plurality of modified Kalman filters (rather than a conventional IMM filter using regular Kalman filters). It should also be noted that a conventional IMM filter does not include thresholding, unlike the present disclosure. The example implementation disclosed herein computes the innovation as a combination of innovations from the plurality of modified Kalman filters, with the weights depending on the user state. One skilled in the art would understand that the example equations of FIGS. 4A-4B are exemplary and are not intended to be limiting. For example, the equations shown in FIGS. 4A-4B may be adapted to take into account other user states and/or other physiological information, for example by adding state-space equations, calculating the relevant innovations and including the appropriate additional terms in the thresholding.

It should be noted that although the example of FIG. 3B accounts for motion as a binary state (e.g., motion vs. no motion), in other examples the system 300 may be adapted to account for different degrees of motion (e.g., running vs. walking vs. no motion) and to account for the user being in a vehicle (e.g., to avoid misclassifying acceleration while the user is in a car as being user motion), among other possible modifications. For example, in order to account for different degrees of motion, there may be a plurality of motion detectors 308a, each of which is adapted to detect occurrence of user motion at a different threshold. The IMM filter 306 may then be adapted to include modified Kalman filters for each of the motion thresholds. It should also be noted that although the example of FIGS. 3A and 3B provide a binary anxiety indicator (e.g., anxiety detected or no anxiety detected), in other examples the output of the anxiety detection system 300 may be non-binary (e.g., may include multiple levels of anxiety detection at different thresholds such as no anxiety, mild anxiety, moderate anxiety and high anxiety; or may provide anxiety detection along a continuous or analog scale).

Although the present disclosure describes user motion as an example user state that can give rise to non-anxiety-specific physiological arousal, it should be understood that other user states may similarly affect the accuracy of anxiety detection. The present disclosure may be adapted to account for such other user states, for example using context data from different physiological and/or non-physiological sensors, and using different user state detectors (e.g., as described with reference to FIG. 3A).

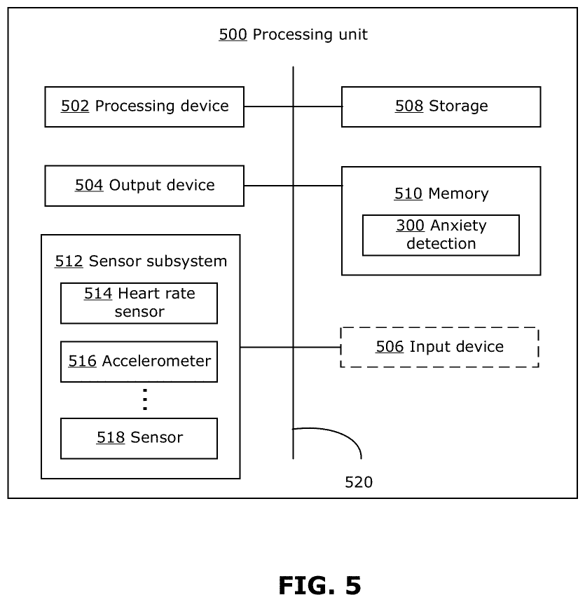

FIG. 5 is a simplified block diagram of an example processing unit 500 in which the disclosed system 300 (e.g., as shown in FIGS. 3A and 3B, and represented by the equations of FIGS. 4A-4B) may be implemented. Although FIG. 5 shows a single instance of each component, there may be multiple instances of each component in the processing unit 500.

The processing unit 500 may be any suitable computing device, such as a portable electronic device, which may be a handheld electronic device (e.g., a mobile phone, a smartphone, a tablet) or a wearable electronic device (e.g., computerized eyeglasses or computerized wrist devices). Such a device may be carried or worn by the subject during daily activities and may thus be able to provide real-time monitoring of the anxiety level of the subject, as well as being able to provide real-time feedback to the subject and/or clinician about the arousal state of the subject. In some examples, the present disclosure may be implemented in conventional portable electronic devices and using conventional physiological sensors. In some examples, some or all computer-executable instructions for implementing the system 300 may be stored externally from the electronic device (e.g., in an external centralized server, in a distributed network, or accessible via cloud computing).

In some examples, the processing unit 500 may include any suitable off-the-shelf wearable device with built-in physiological sensor (e.g., a wearable activity tracker) and any suitable consumer portable electronic device. A downloadable software application (also referred to as an app) for implementing the disclosed method may be installed onto the processing unit 500. The software may be updated as appropriate to incorporate new relaxation techniques, different numbers and/or types of physiological sensors, and/or new feedback techniques, for example.

The processing unit 500 may include one or more processing devices 502, such as a processor, a microprocessor, an application-specific integrated circuit (ASIC), a field-programmable gate array (FPGA), a dedicated logic circuitry, or combinations thereof. The processing unit 500 may include one or more output devices 504 (e.g., a display, a speaker, a tactile/vibration mechanism and/or a light), which may provide feedback (e.g., to the subject and/or clinician) based on the detected anxiety level. The processing unit 500 may optionally include one or more input devices 506 (e.g., a keyboard, a mouse, a microphone, a touchscreen, and/or a keypad), which may receive input (e.g., command instructions) from a user. Although not shown, in some examples the processing unit 500 may include components (e.g., network interfaces) to enable wired or wireless communication.

The processing unit 500 may also include one or more storage units 508, which may include a mass storage unit such as a solid state drive, a hard disk drive, a magnetic disk drive and/or an optical disk drive. The processing unit 500 may include one or more memories 510, which may include a volatile or non-volatile memory (e.g., a flash memory, a random access memory (RAM), and/or a read-only memory (ROM)). The non-transitory memory(ies) 510 may store instructions for execution by the processing device(s) 502, such as to implement an example of the disclosed anxiety detection system 300. The memory(ies) 510 may also store databases of relaxation techniques, a subject's history of anxiety, a log of occurrences of detected anxiety, and other information about the subject, as well as patterns of physiological activity in the subject or larger populations, for example.

The memory(ies) 510 may include other software instructions, such as for implementing an operating system and other applications/functions. In some examples, one or more data sets and/or modules may be provided by an external memory (e.g., an external drive in wired or wireless communication with the processing unit 500) or may be provided by a transitory or non-transitory computer-readable medium. Examples of non-transitory computer readable media include a RAM, a ROM, an erasable programmable ROM (EPROM), an electrically erasable programmable ROM (EEPROM), a flash memory, a CD-ROM, or other portable memory storage. Some or all of the instructions and/or data described above as being stored in the memory(ies) 510 and/or storage unit(s) 508 may be stored externally (e.g., in an external centralized server, in a distributed network, or accessible via cloud computing) and accessible by the processing unit 500.

The processing unit 500 in this example includes a sensor subsystem 512, which includes one or more sensor units, in this example a heart rate sensor 514, an accelerometer 516 and one or more other sensors 518 (generically referred to as the sensor subsystem 512). The sensor subsystem 512 may include sensors positionable on or near the subject for obtaining physiological data. Other sensors for obtaining context data may be positional on or near the subject, or may not need to be close to the subject. Any suitable sensor(s) may be used, such as wearable heart rate sensors or electrodes. Although shown as being part of the processing unit 500, in some examples one or more sensors of the sensor subsystem 512 may be external to the processing unit 500 and may communicate data via signals (e.g., wired or wireless signals) to the processing device 502.

There may be a bus 520 providing communication among components of the processing unit 500, including the processing device(s) 502, output device(s) 504, optional input device(s) 506, storage unit(s) 508, memory(ies) 510 and the sensor subsystem 512. The bus 520 may be any suitable bus architecture including, for example, a memory bus, a peripheral bus or a video bus.

The example processing unit 500 may provide feedback to the subject and/or a clinician about the arousal state of the subject, via the output device 504. The anxiety indication 312 outputted by the anxiety detection system 300 may be used to determine the type of output to be provided. For example, when the anxiety indication 312 indicates that the subject is at or close to experiencing anxiety, the processing device 502 may cause the output device 504 to provide visual and/or audio feedback to indicate the subject is experiencing anxiety and/or to enable relaxation or desensitization.

The type of output that is provided, based on the anxiety indication 312, is not limited. For example, the output may include audio output, tactile output, visual output, or a combination of these. The output may be directed to the clinician, in which case the output may simply provide information about the presence or absence of anxiety. Additionally or alternatively, the output may be directed to the subject and/or care-giver, in which case the output may be designed to help the subject return to a state of lessened or no anxiety. Such further output may be in the form of visual or audio suggestions of relaxation techniques, or distractions (all of which may be pre-stored in the memory(ies) 510), for example. The subject's anxiety level may be monitored (using the output from the anxiety detection system 300) while this output is provided, so that the success of the relaxation/distraction technique may be determined.

In some examples, a log of the subject's anxiety state may be created and the log may be stored in the memory 510 and/or outputted to be stored in an external memory. Information in the log may include the subject's anxiety level (as represented by the anxiety indicator) and the associated context, for example. Information included in such a log may be useful to help the subject and/or clinician to identify anxiety triggers and successful relaxation techniques, for example.

In some examples, information about the subject's anxiety state may be used as a measurement or representation of the engagement of the subject in an activity (e.g., user engagement in a game). The anxiety indication 312 may be used as feedback for automatic, semi-automatic or manual adjustment of the activity (e.g., increasing or decreasing difficulty of the game) in order to increase or decrease user engagement, for example.

### Example Study

An example of the disclosed system 300 was evaluated in a study using data collected from a sample of children and youth with a diagnosis of ASD (n=15). All participants had a clinical diagnosis of ASD, supported by the gold-standard instruments namely, the autism diagnostic observation schedule (ADDS) and the autism diagnostic interview—revised (ADI-R). Participants were between the ages of 8 and 16 and had full-scale IQ scores greater than 50. Participants using beta-blockers were excluded from the study as these medications have a significant effect on physiological arousal. Participants' IQ was assessed using the Wechsler Abbreviated Scale of Intelligence (WASI), and ASD symptom severity was characterized using the Social Communication Questionnaire (SCQ). Table I details the characteristics of the sample:

TABLE I

| Measure | Mean (SD) | |
|---|---|---|
| Age | 14 | (1.77) |
| Sex (Male:Female) | 9:6 | |
| Full-scale IQ | 89.9 | (15.40) |
| SCQ scoare | 20 | (7.37) |

The Shimmer™ 2r sensor from Shimmer technologies was used to collect physiological and motion data. The sensor consisted of an ECG acquisition system, accelerometer, and wireless bluetooth capabilities that allowed for untethered, wireless communication to a data collection computer. Gel-electrodes where attached to four loci on the chest: the right and left arm electrodes placed in the first intercostal space and on the midclavicular line, and the right and left leg electrodes placed on the midclavicular line inferior to the tenth rib. The modified four-electrode placement was used to reduce the denigrative effect of motion artefacts on the signal-to-noise ratio of the ECG signal. The Shimmer 2r was attached to the chest to allow measurement of changes in torso acceleration along the x, y, and z planes using the on-board accelerometer. The accelerometer and ECG signals were sampled at 250 Hz.

### Experimental Protocol

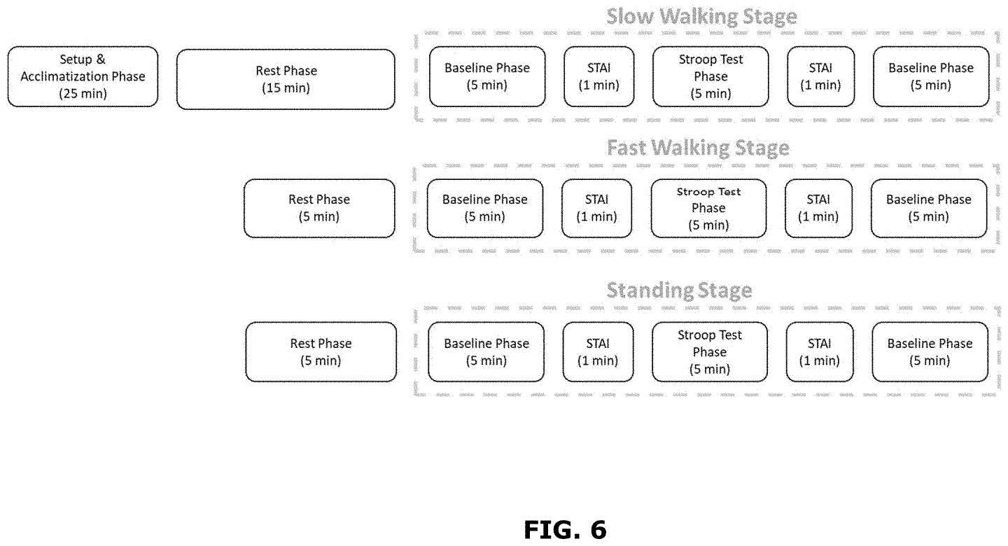

As shown in FIG. 6, the testing session consisted of three stages during which the participants were asked to either stand, slow walk, or walk a comfortable speed (fast walk) on a treadmill. Prior to the start of each stage, a resting baseline was captured while the participant was seated, and engaged in a 5-minute movie clip. The first resting phase was used to initialize system parameters. The treadmill was then set to speeds appropriate to the activity level being tested: during the standing stage, the treadmill was not turned on, slow walking was set to the first speed setting supported by the treadmill, and during the fast walking, the treadmill was set to a comfortable speed that aligned with the participant's gait.

During each of the stages, participants completed a baseline and stressor activity. The baseline phase consisted of watching a 5-minute clip from BBCs Planet Earth 2. Clips were chosen specifically to not include scenes that can induce anxiety, and rapid changes in music, violence, or frightening scenes were excluded from any of the clips shown to the participants. The Stroop Colour-Word Interference test was chosen as the stressor activity to induce anxiety-related arousal. This task has previously been used to induce anxiety in many studies [12], [25]-[28]. During this test, participants were asked to name the font colour of the word that is being displayed on the screen. The words were chosen at random, as are the font colour, from a list of colours: blue, red, green, purple, and yellow. The Stroop tests were five minutes in length, and were divided into five, one-minute blocks. Blocks 1, 3 and 5 were set to present words at two second intervals, and blocks 2 and 3 at 1.25-second intervals. The congruent section (matching colour name and print colour) were made up of blocks 1, and 5, while the rest of the blocks were made up of the in-congruent section (conflicting colour name and print colour). This protocol has been previously used in studies eliciting anxiety responses in children with ASD [12].

Sensitivity, specificity, and accuracy were to evaluate the performance of the filter in classifying baseline and condition states. Each of the metrics were defined as:

$$Sensitivity = \frac{TP}{TP + FN} , \\ Specificity = \frac{TN}{TN + FP} , \\ Accuracy = \frac{Sensitivity + Specificity}{2}$$

where true positive TP, true negative TN, false positive FP, and false negative FN are calculated relative to ground truth signals for motion and arousal. The ground truth for motion detection was determined to be motion when the participant walked and no motion otherwise.

For arousal, determining the ground truth is challenging due to difficulties in obtaining reliable self- or parent reports to gauge the emotional states of children with ASD. Therefore, the approach of [12] was followed, and intervals with increases of two or more beats in heart rate relative to the preceding baseline mean were designated as arousal intervals.

The heart rate response to the motion and anxiety tasks were characterized. In addition, the performance of the disclosed anxiety detection system 300 as well as its sensitivity to its parameters is evaluated.

### Characterisation of Heart Rate Response

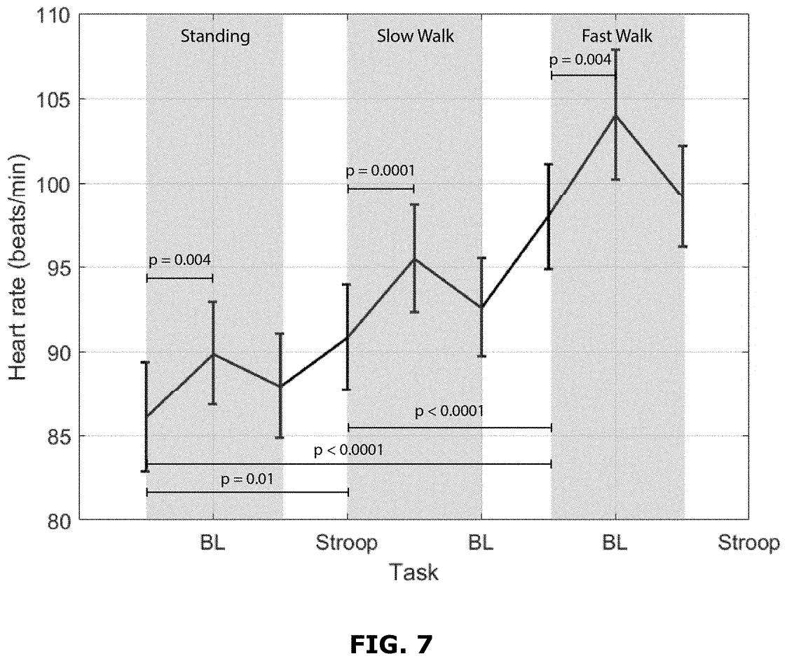

The average heart rate across all participants is presented in FIG. 7 for all study tasks. In FIG. 7, BL indicates when the participants are performing baseline task, and Stroop indicates the color-word interference task. Bars represent standard error. The effect of task on heart rate was analysed using repeated measures linear regression analysis. In particular, heart rate differences between the motion and arousal conditions were analyzed. Based on Bonferroni correction for six comparisons, a significance level of 0.01 was used.

Effect of motion on heart rate: The analyses showed significantly increased heart rate during the fast walking baseline compared to slow walking and standing baselines (fast walking—standing: estimated difference=11.89±2.03 beats/min, p<0.0001; fast walking—slow walking: estimated difference=7.14±1.61 beats/min, p<0.0001).The difference in heart rate between standing and slow walking baselines was also significant (slow walking—standing: estimated difference=4.75±1.79 beats/min, p=0.01).

Effect of anxiety on heart rate: There was a significant increase in heart rate during the Stroop task compared to the baseline for all three motion conditions (standing—baseline: estimated difference=3.79±1.29 beats/min, p=0.004; slow walking—baseline: estimated difference=4.62±1.17 beats/min, p=0.0001; standing—baseline: estimated difference=6.05±2.08 beats/min, p=0.004).

### Motion Detection

The effect of system parameters on the performance of the motion detector 308a was examined. In particular, the effect of the width of the acceleration feature smoothing window, innovation window width, and the detection threshold on sensitivity, specificity, and accuracy were examined.

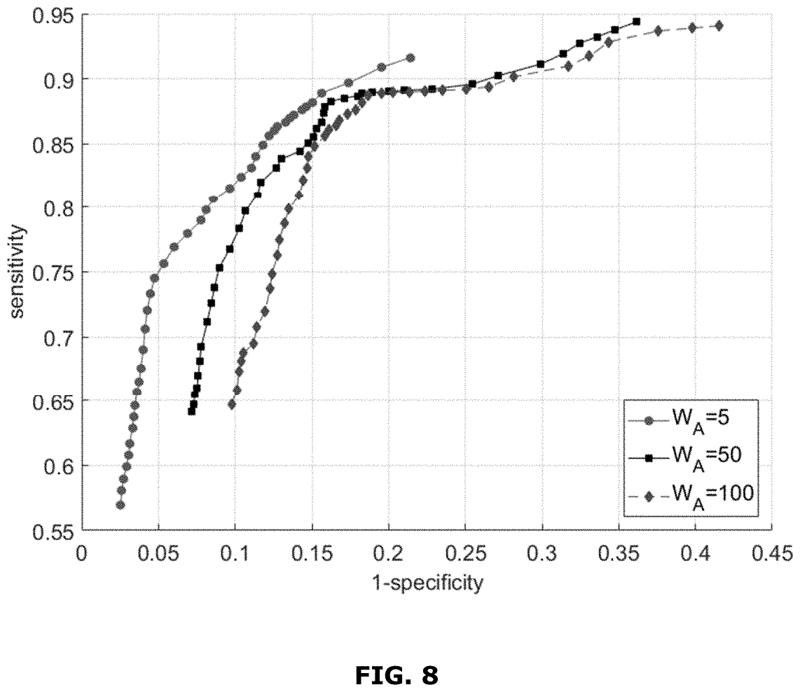

Acceleration smoothing window length: The parameter w_{A} is used to compute the acceleration feature (Equation 1). FIG. 8 depicts the effect of this parameter on algorithm performance. As seen, algorithm performance in this instance was found to be optimized for W_{A}=5.

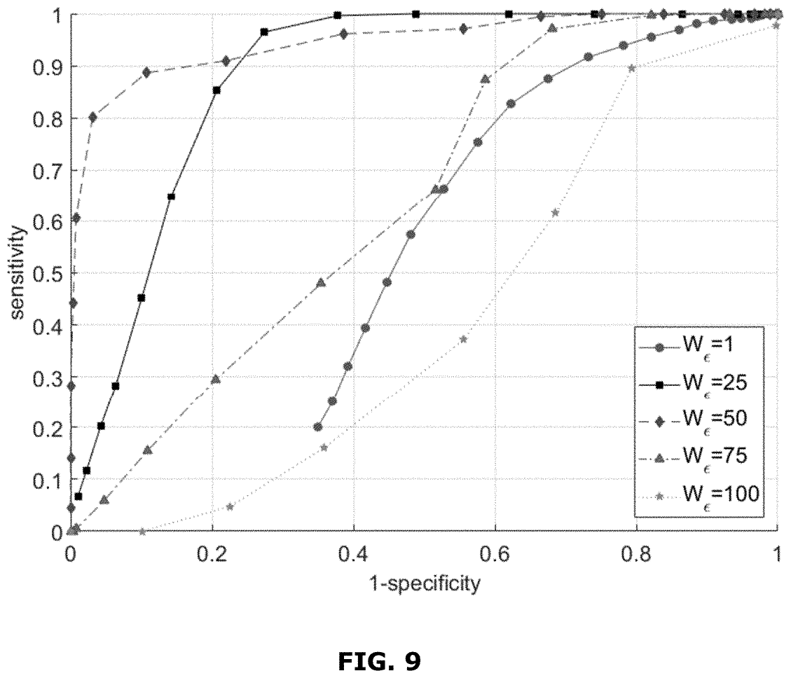

Innovation window width: The window width W_{ϵ} is used to smooth the innovation time-series used for thresholding. The effect of this parameter on system performance is shown in FIG. 9. As seen, in this instance, the value of W_{ϵ}=50 was found to maximize performance of the motion detector 308a.

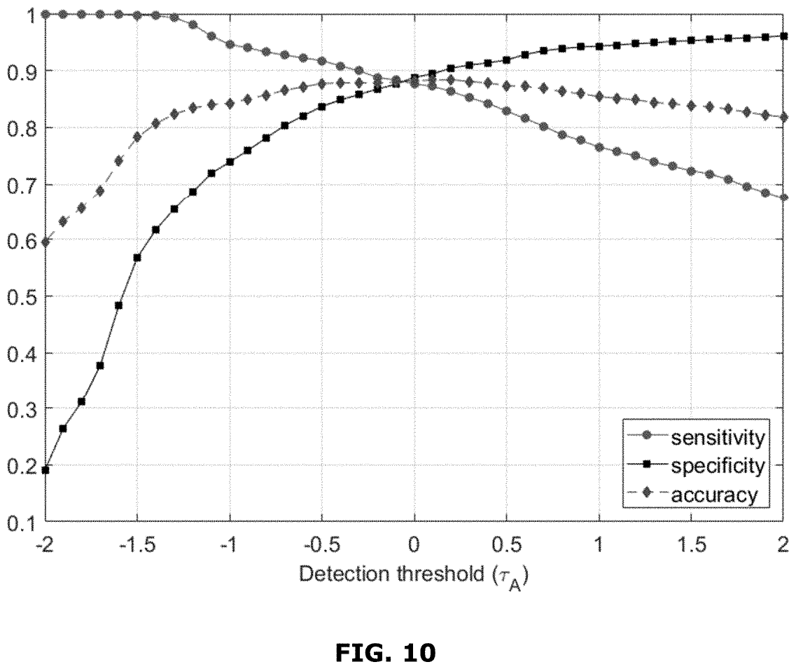

Detection threshold: The threshold τ_{A} is used in the motion detector 308a. FIG. 10 shows the effect of this parameter on motion detection performance and, in this instance, a value of τ_{A}=0 was found to be preferrable.

### Anxiety Detection

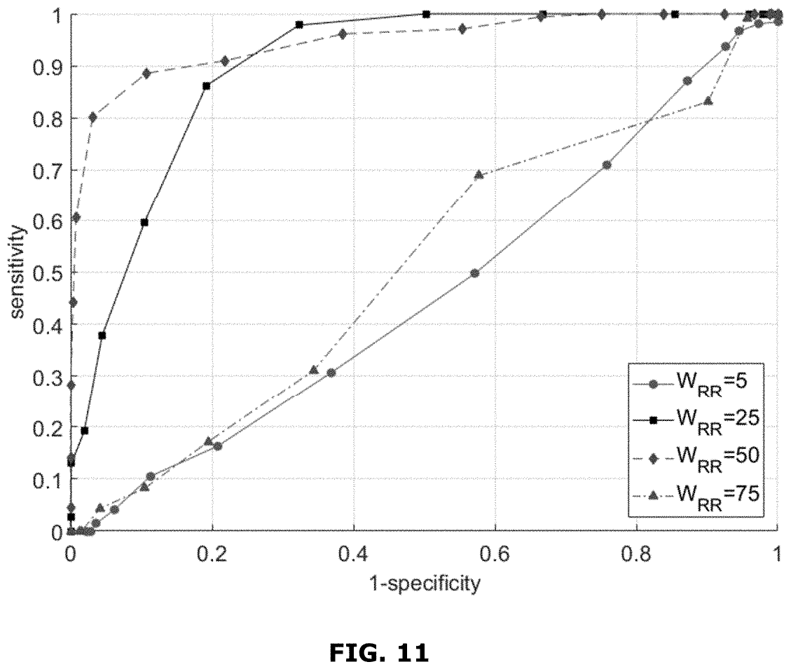

RR smoothing window length: A moving average window of length W_{RR} was used to compute the slowly varying RR trend for the anxiety detector 310. FIG. 11 depicts the effect of this parameter on filter performance and in this instance a value of W_{RR}=50 was found to provide optimal performance.

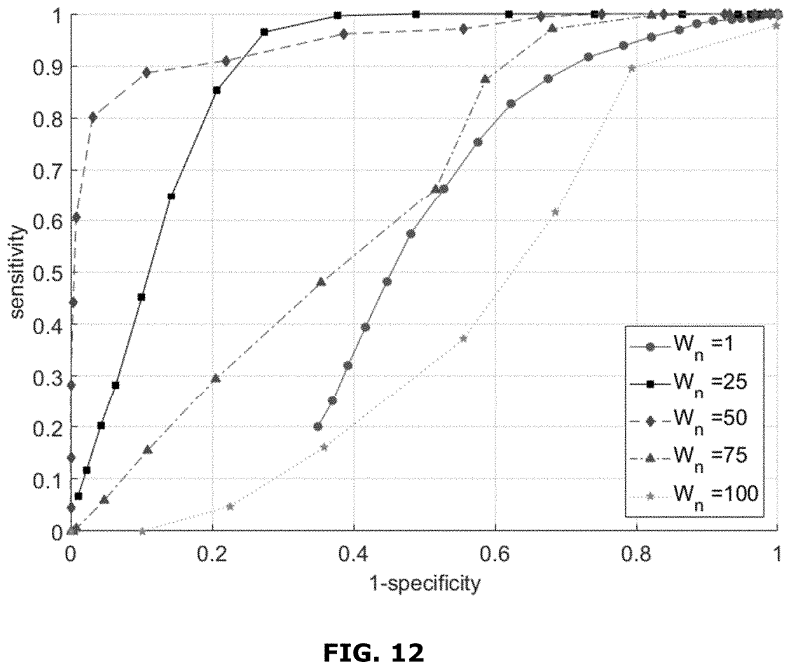

Innovation Window length: FIG. 12 shows the effect of Wₙ on performance. This parameter is the innovation smoothing window length. In this instance, the value of Wₙ=50 was found to provide the best performance on this example dataset.

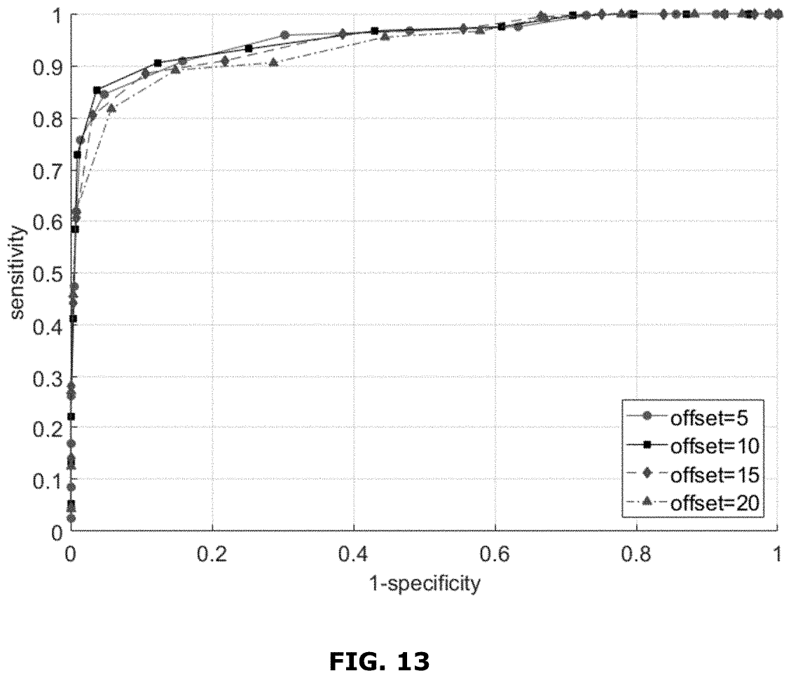

Offset: The offset parameter specifies the difference in initial state means between the filers matched to rest and motion. FIG. 13 shows the effect of this parameter on algorithm performance. The figure suggests that the anxiety detector 310 is not highly sensitive to the initialization offset. An offset of 10 beats/minute is chosen for the remaining analyses.

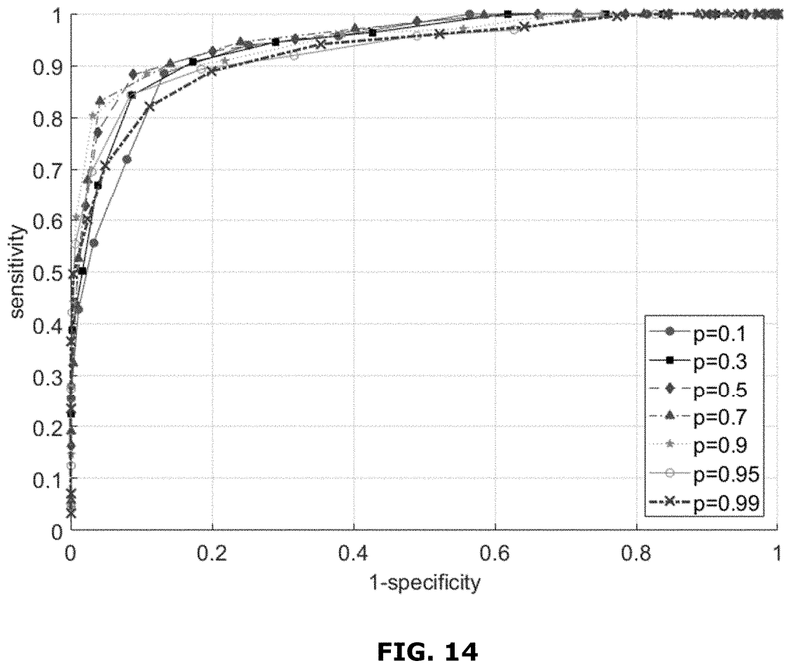

Transition probabilities: The effect of transition probabilities on algorithm performance was examined (FIG. 14). These values impact the computation of mode probabilities in the algorithm (motion versus rest). As seen, in this instance, optimal algorithm performance was found to be achieved with a relatively wide range of parameter values between 0.5 and 0.9.

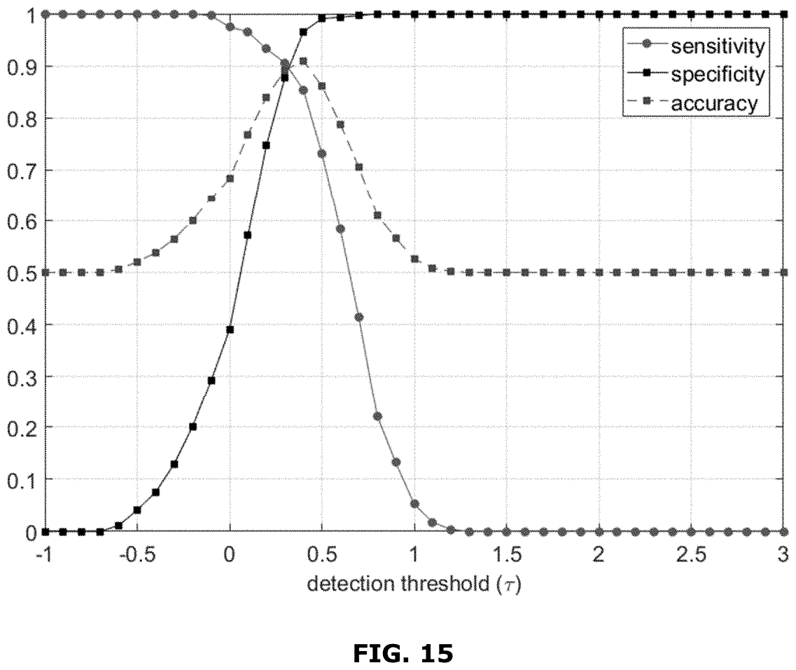

Detection threshold: FIG. 15 depicts the effect of threshold τₐₙₓ on the sensitivity, specificity and accuracy of the anxiety detector 310, suggesting that the best results, in this instance, are obtained with for τₐₙₓ=0.5.

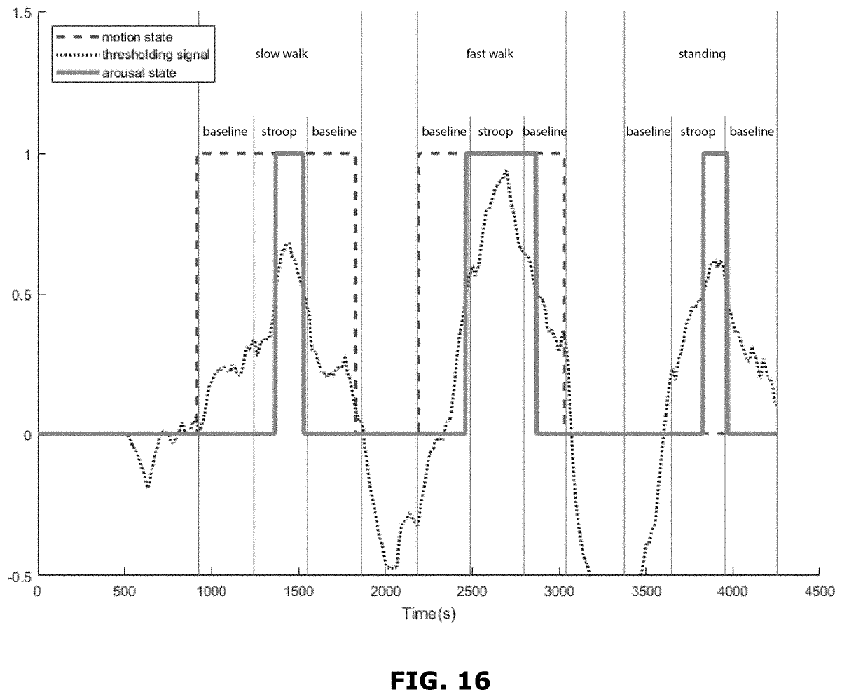

FIG. 16 provides an example illustrating an example operation of the disclosed anxiety detection system. In particular, the internal signal for detected user state (1=user motion state; 0=no motion state), the internal thresholding signal, and outputted anxiety indication signal (1=anxiety-related arousal detected; 0=anxiety-related arousal not detected) are shown. In this example, anxiety-related arousal is detected when the thresholding signal exceeds 0.5.

In various examples, the present disclosure describes anxiety detection methods and systems, which can detect anxiety with accuracy in different user states including states (e.g., user motion) that may cause physiological arousal that is not specific to anxiety. In particular, the disclosed methods and systems provide an unsupervised algorithm for anxiety detection. Another unsupervised approach, using a modified Kalman filter, has been described in U.S. Pat. No. 9,844,332, having a common inventor to the present application.

Table II compares the performance of an example of that previous algorithm to an example of the presently disclosed method. Parameters of both algorithms were optimized to obtain the best accuracy. As seen, the presently disclosed method provides a significant advantage in terms of achieved accuracy, and especially with regards to improving algorithm specificity. Performance was compared under subject conditions of standing still (SS), slow walking (SW) and fast walking (FW). The performance averaged over all conditions was also compared. In particular, 16% and 22% improvement in specificity is achieved by the presently disclosed method for the slow walking and fast walking conditions, respectively.

TABLE II

| Approach | Condition | Accuracy | Sensitivity | Specificity |
|---|---|---|---|---|
| Modified | SS | 0.82 | 0.74 | 0.89 |
| Kalman | SW | 0.82 | 0.85 | 0.79 |
| filter | FW | 0.87 | 0.97 | 0.77 |
| [12] | All | 0.84 | 0.82 | 0.85 |
| Example | SS | 0.87 | 0.78 | 0.95 |
| disclosed | SW | 0.93 | 0.90 | 0.95 |
| method | FW | 0.99 | 0.99 | 0.99 |
| | All | 0.91 | 0.85 | 0.97 |

These results demonstrate that the example disclosed method is able to detect anxiety responses to an anxiety task (Stroop task) with accuracy greater than 85% during three motion scenarios: standing still, slow walking, and fast walking. This represents a significant improvement compared to the state-of-the-art anxiety detection systems, especially with regards to specificity of anxiety detection.

The disclosed methods and systems thus may be used to provide objective feedback indicating anxiety level, which may be useful for populations who have difficulties with self-awareness and communication of these states, such as children with a diagnosis of ASD. Other populations may also benefit from examples disclosed herein. The disclosed methods and systems may be implemented in consumer devices (e.g., wearable activity monitors), may be used for general wellness monitoring, may be used in a clinical setting (e.g., for treatment of anxiety or desensitization treatments), or other such applications. Further, the disclosed methods and systems may be adapted for other user states (e.g., hot/cold, sleeping/awake, etc.), which may help to enable accurate anxiety detection in everyday situations and naturalistic settings.

The disclosed methods and systems may be integrated into a larger system, such as a virtual reality platform, for anxiety treatment and/or desensitization, for example.

In the example study discussed above, the performance of the example disclosed method was examined with respect to variations in certain parameters. While certain parameter values (e.g., smoothing window lengths) were found to be optimal in this instance, it should be understood that they are exemplary and are not intended to be limiting. Further, it should be understood that although certain parameters were examined in the case were user motion is of concern, other parameters may be relevant for other user states. One skilled in the art would understand how to select and adjust parameters, for example using routine trial-and-error or through empirical methods.

The disclosed methods and systems use a modular approach where additional filter models and user state detectors can be added to integrate other user states that may be associated with ANS arousal. The disclosed methods and systems use an unsupervised approach for anxiety detection, which may avoid the expense and training required for supervised learning approaches.

In an example disclosed herein, an anxiety detection system is provided in which user motion is taken into account, to enable accurate detection of anxiety both when the user is in motion and when there is no user motion. The example anxiety detection system combines information from an accelerometer with information from a heart rate monitor, resulting in a system that is resilient against motion, and avoids false positives.

The disclosed methods and systems may be implemented in wearable devices that can provide a real-time and objective feedback to a subject and/or a clinician about the subject's arousal state. In particular, the disclosed methods and systems may enable anxiety detection in different user states, such as user motion, which may facilitate the translation of the technology from laboratory environments to everyday settings.

In some examples, the present disclosure may be embodied in the form of instructions accessible by an electronic device via cloud computing. In some examples, the present disclosure may be embodied in the form of an application programming interface (API) (e.g., at a server) accessible by an electronic device.

Although the present disclosure is described, at least in part, in terms of methods, a person of ordinary skill in the art will understand that the present disclosure is also directed to the various components for performing at least some of the aspects and features of the described methods, be it by way of hardware components, software or any combination of the two. Accordingly, the technical solution of the present disclosure may be embodied in the form of a software product. A suitable software product may be stored in a pre-recorded storage device or other similar non-volatile or non-transitory computer readable medium, including DVDs, CD-ROMs, USB flash disk, a removable hard disk, or other storage media, for example. The software product includes instructions tangibly stored thereon that enable a processing device (e.g., a personal computer, a server, or a network device) to execute examples of the methods disclosed herein.

The present disclosure may be embodied in other specific forms without departing from the subject matter of the claims. The described example embodiments are to be considered in all respects as being only illustrative and not restrictive. Selected features from one or more of the above-described embodiments may be combined to create alternative embodiments not explicitly described, features suitable for such combinations being understood within the scope of this disclosure.

All values and sub-ranges within disclosed ranges are also disclosed. Also, although the systems, devices and processes disclosed and shown herein may comprise a specific number of elements/components, the systems, devices and assemblies could be modified to include additional or fewer of such elements/components. For example, although any of the elements/components disclosed may be referenced as being singular, the embodiments disclosed herein could be modified to include a plurality of such elements/components. The subject matter described herein intends to cover and embrace all suitable changes in technology.

1. A system for providing output based on detection of anxiety in a subject, the system comprising:
an output device for providing output dependent on an anxiety indication, the anxiety indication representing a current or expected level of anxiety in the subject;
a memory;
a processor coupled to the output device and the memory;
the processor configured to execute computer-executable instructions to cause the system to:
receive at least one physiological signal, from a first sensor, the physiological signal representing physiological information from the subject;
receive at least one context signal;
implement a user state detector to determine a current user state from a plurality of possible user states, based on the at least one context signal;
implement an interactive multiple model (IMM) filter to determine, using the physiological signal, a respective statistical prediction of anxiety in each of the plurality of possible user states; and
implement an anxiety detector to output the anxiety indication, based on a weighting of the respective statistical predictions using the determined current user state.

2. The system of claim 1, wherein the instructions, when executed, further cause the system to:
implement a feature extractor to:
extract the at least one physiological feature from the at least one physiological signal, the at least one physiological feature being affected by the level of anxiety in the subject; and
extract the at least one context feature from the at least one context signal, the at least one context feature being relevant to determination of the current user state;
wherein the user state detector determines the current user state based on the at least one context feature extracted from the at least one context signal; and
wherein the IMM filter determines the respective statistical predictions based on the at least one physiological feature extracted from the at least one physiological signal.

3. The system of claim 2, wherein the instructions, when executed, further cause the system to implement the feature extractor to:
extract the at least one physiological feature by calculating a trend using a first defined smoothing window length; and
extract the at least one context feature by calculating a moving standard deviation using a second defined smoothing window length.

4. The system of claim 1, wherein the at least one physiological signal comprises a heart rate signal, wherein the at least one context signal comprises an acceleration signal, and wherein the plurality of possible user states includes a first user state where the user is in motion and a second user state where the user is not in motion.

5. The system of claim 4, further comprising:
a heart rate monitor for generating the heart rate signal; and
an accelerometer for generating the acceleration signal.

6. The system of claim 1, wherein the instructions, when executed, further cause the system to implement the user state detector to:
determine the current user state using a modified Kalman filter.

7. The system of claim 1, wherein the instructions, when executed, further cause the system to implement the IMM filter to:
determine the respective statistical prediction of anxiety using a respective modified Kalman filter matched to each respective possible user state.

8. The system of claim 1, wherein at least one of the at least one context signal is received from a context sensor of the system.

9. The system of claim 1, wherein at least one of the at least one context signal is received from an external system.

10. The system of claim 1, wherein the output device is a display screen and the provided output is a visual output that is responsive to the current or expected level of anxiety in the subject.

11. The system of claim 1, wherein the system is implemented in a portable electronic device.

12. The system of claim 1, wherein the system is implemented in a wearable electronic device.

13. The system of claim 1, wherein the system is implemented in a virtual reality device.

14. The system of claim 1, wherein the instructions are executable by the processor via cloud computing.

15. The system of claim 1, wherein the instructions are executable by the processor via an application programming interface (API) on a server.

16. A method, implemented in an electronic device, for providing output based on detection of anxiety in a subject, the method comprising:
receiving at least one physiological signal, from a first sensor coupled to the electronic device, the physiological signal representing physiological information from the subject;
receiving at least one context signal;
implementing, in the electronic device, a user state detector to determine a current user state from a plurality of possible user states, based on the at least one context signal;
implementing, in the electronic device, an interactive multiple model (IMM) filter to determine, using the physiological signal, a respective statistical prediction of anxiety in each of the plurality of possible user states;
implementing, in the electronic device, an anxiety detector to output an anxiety indication, based on a weighting of the respective statistical predictions using the determined current user state, the anxiety indication representing a current or expected level of anxiety in the subject; and
providing output, via an output device of the electronic device, dependent on the anxiety indication.

17. The method of claim 16, further comprising implementing, in the electronic device, a feature extractor to:
extract the at least one physiological feature from the at least one physiological signal, the at least one physiological feature being affected by the level of anxiety in the subject; and
extract the at least one context feature from the at least one context signal, the at least one context feature being relevant to determination of the current user state;
wherein the user state detector determines the current user state based on the at least one context feature extracted from the at least one context signal; and
wherein the IMM filter determines the respective statistical predictions based on the at least one physiological feature extracted from the at least one physiological signal.

18. The method of claim 17, wherein the at least one physiological signal comprises a heart rate signal received from a heart rate sensor coupled to the electronic device, wherein the at least one context signal comprises an acceleration signal received from an accelerometer coupled to the electronic device, and wherein the plurality of possible user states includes a first user state where the user is in motion and a second user state where the user is not in motion.

19. The method of claim 16, wherein the user state detector determines the current user state using a modified Kalman filter.

20. The method of claim 16, wherein the IMM filter determines the respective statistical prediction of anxiety using a respective modified Kalman filter matched to each respective possible user state.
